(19) United States
(12) Patent Application Publication
(10) Pub. No.: US 2012/0149950 A1
(43) Pub. Date: Jun. 14, 2012

(54) PLANT FOR THE HOMOGENEOUS OXIDATION OF METHANE-CONTAINING GAS AND PROCESS FOR THE OXIDATION OF METHANE-CONTAINING GAS

(76) Inventors: Valer'jan Nikolaevich Blinichev, Ivanovo (RU); Rafis Faizovich Kadyrov, Upkankul (RU); Oleg Vjacheslavovich Chagin, Ivanovo (RU)

(21) Appl. No.: 13/391,256

(22) PCT Filed: Nov. 16, 2009
(86) PCT No.: PCT/RU2009/000625

§ 371 Date: Feb. 17, 2012

(30) Foreign Application Priority Data
Aug. 19, 2009 (RU) 2009131339

(51) Int. Cl. C07C 27/00; B01J 19/00

(52) U.S. Cl. 568/959; 422/187

(57) ABSTRACT

The invention relates to the process for homogenous oxidation of methane-containing gas comprising feeding of methane-containing gas preheated to 430-450° C. to at least three sequentially installed oxidation reactors, wherein each reactor, with the exception of the last one, is independently connected to waste-heat boilers, the reactors are fed with oxygen for oxidation of methane-containing gas, wherein the gas mixture temperature is increased to 540-560° C. with subsequent rapid quenching-cooling of the gas mixture to 440-450° C. in waste-heat boilers where steam is formed which is fed to a rectification tower for separation of end products, from the last reactor the reaction mixture is fed to a separator, from the separator the liquid phase is fed to the rectification stage where rectified methanol, ethanol and formaldehyde are produced, and the gas phase is fed for cleaning it of SO2, CO and CO2, and in the process, partial purging of the circulation cycle is performed to remove inert gases, and after cleaning and purging the cycle is completed by replenishing the gas-phase/cycled gas with the original methane-containing gas and re-feeding, the resulting gas to the reactor. The invention makes it possible to increase efficiency, increase product yield and improve the environmental situation.

### CROSS-REFERENCE TO RELATED APPLICATION

This application is a national stage application and claims the benefit of the priority filing date in PCT/RU2009/000625 referenced in WIPO Publication WO/2011/021955. The earliest priority date claimed is Aug. 19, 2009.

### FEDERALLY SPONSORED RESEARCH

None

### SEQUENCE LISTING OR PROGRAM

None

### STATEMENT REGARDING COPYRIGHTED MATERIAL

Portions of the disclosure of this patent document contain material that is subject to copyright protection. The copyright owner has no objection to the facsimile reproduction by anyone of the patent document or the patent disclosure as it appears in the Patent and Trademark Office file or records, but otherwise reserves all copyright rights whatsoever.

### BACKGROUND

The invention relates to the petrochemical industry, and particularly, to a plant for homogenous oxidation of methane-containing gas and to a process for homogenous oxidation of methane-containing gas.

There are large reserves of natural gas, which is an ecologically clean fuel, but in many cases, in particular, in transportation, direct use of a gaseous energy source is inconvenient. The problem is solved by converting natural gas to a more universal liquid fuel, such as methanol, which is feedstock for a number of chemical production processes.

Known are a number of processes for converting methane to methanol. Widely used in the industry is methane steam conversion to synthesis gas (a mixture of CO and H2) with subsequent catalytic conversion of the latter to methanol (Karavaev, M. M., Leonov, V. E. et al., “ [symbol] [symbol] [symbol] ” [Synthetic Methanol Production Processes], Moscow, “Khimiya” Publishing House, 1984, p. 72-125). However, this process has a number of significant disadvantages, for example, equipment complexity, strict requirements for gas cleanness, high energy consumption for production and cleaning of synthesis gas, a large number of intermediate stages of the process, and unprofitability of small and medium production facilities with a capacity of under 2000 t/day.

Currently, most interests lie in the direct gas-phase oxidation of methane to methanol at high pressures, bypassing the phase of synthesis gas production. The process is conducted in tubular reactors at pressures of up to 10 MPa and at a temperature of 400-450° C. with relatively low original concentrations of oxygen, with subsequent cooling of the gas-liquid mixture, with a separation of liquid products, and with an extraction of methanol from liquid products by rectification (Arutyunov, V. S., and Krylov, O. V., “ [symbol] [symbol] [symbol] ” [Oxidative Transformations of Methane], Moscow, “Nauka” Publishing House, 1998, p. 130-145). However, the low rate of methane conversion during one pass through the reactor does not exceed 3-5% and, accordingly, the low methanol yield inhibits practical implementation of the process for producing methanol by direct methane oxidation.

Methane-containing gas refers to gases containing at least 50% of CH4, as well as flare gases burned in oil and gas fields. Depending on the deposit of oil containing seams, methane concentration in gas flares currently vary from 30% to 86% (Arutyunov, V. S., and Krylov, O. V., “ [symbol] [symbol] [symbol] ” [Oxidative Transformations of Methane], Chemical Physics Institute, RAN [Russian Academy of Sciences], Moscow, “Nauka” Publishing House, 1998, 361, pp.)

A plant for homogenous oxidation of methane-containing gas makes it possible to increase the efficiency and cost effectiveness of gas processing. This is achieved by simultaneously using waste-heat boilers for performing a rapid quenching of the reaction mixture from reactors (for homogenous oxidation of methane-containing gas). This is also achieved by absorbers that clean cycled gas of impurities and manufacturing process byproducts, such as SO2, CO and CO2.

Furthermore, the efficiency and cost effectiveness of gas processing is achieved by a pipeline which is additional and connects waste-heat boilers, as well as one of the heat exchangers (which cools cycled gas coming out from the stage of oxidation of the original methane-containing gas), with an apparatus for separating (rectification) homogenous oxidation products. The proposed process also makes it possible to achieve a similar effect. It too uses the above innovations, but with a refinement. Before cycled gas is fed to the first reactor, it is heated to 430-450° C. After cooling, condensation and separation of reaction products, the cycled gas is cleaned of SO2, CO and CO2.

Known is a process for the production of methanol comprising a separate feeding of hydrocarbon-containing gas (preheated to 200-500° C. at a pressure of 2.5-15 MPa) and oxygen-containing gas into a mixing chamber. This is followed by partial methane oxidation with oxygen concentration of 1-4% by volume, with an additional introduction of reagents (a metal-oxide catalyst, higher gaseous hydrocarbons or oxygen-containing compounds, and a cold oxidizer) to the reactor reaction zone. Then the reaction mixture is cooled in a heat exchanger. Methanol is separated from liquid reaction products in a separator, and exit gaseous reaction products are fed to the reactor inlet (RU, 2049086 C 1, C 07 C 31/04, Nov. 27, 1995). The process's disadvantage is the need to use a catalyst or additional reagents, as well as a strong heating of reactant gases, which reduces methanol yield and increases the probability of soot formation.

Known is the process for the production of methanol comprising a separate feeding of hydrocarbon-containing gas (natural gas or methane) and oxygen-containing gas (air or oxygen) into a mixer. This is followed by feeding the mixture to an inert reactor, partial gas-phase oxidation of the hydrocarbon-containing gas in the reactor (at a pressure of 1-10 MPa for 2-1000 s at a temperature of 300-5000 C without a catalyst, with oxygen content of 2-20% by volume), separating methanol in a condenser from liquid reaction products, and returning exit reaction gases (containing unconverted methane for mixing with the original hydrocarbon-containing gas) to the first reactor or into the second reactor connected sequentially to the first reactor (GB, 2196335 A, C 07 C 31/04, Apr. 27, 1988). The process's disadvantage is a long reaction time which limits the reactor's methanol capacity.

The plant for production of methanol comprises the following, which are sequentially installed and connected by means of pipelines: a mixing chamber connected to separate sources of hydrocarbon-containing gas, and air or oxygen; a reactor made of an inert material with heating elements for the partial oxidation of methane in a mixture fed into the reactor at a positive pressure; a condenser and a separator for separating methanol from reaction products; and a vessel for recycled reaction products, with a pipeline for feeding them to the original hydrocarbon-containing gas or to the mixing chamber (GB, 2196335 A, C 07 C 31/04, Apr. 27, 1988). The process and plant provide a high methanol yield, and 5-15% of methane may react every time it passes through the reactor. However, reagents spend a long time in the reactor, which does not make it possible to ensure the plant's high production rate.

Known is a process for the production of methanol comprising the separate feeding and oxidation of hydrocarbon-containing gas with oxygen-containing gas at 370-450° C. and a 5-20 MPa pressure, with their contact time in the reactor of 0.2-0.22 s, and cooling the heated reaction mixture to 330-340° C. by injecting methanol in the reactor (SU, 1469788 A1, C 07 C 31/04, Nov. 20, 96); or cooling the reaction mixture without intermediate condensation and separation to 380-4000 C in interstage heat exchangers installed in the reactor. The reaction mixture is then fed to 2-3 sequential oxidation stages (SU, 1336471 A1, C 07 C 31/04, Sep. 27, 96). The process' disadvantage is a need for additional consumption and repeated separation of methanol, which leads to an inevitable loss of methanol, or to the installation of additional cooling loops with an additional cooling agent circulating in it.

From RU 2057745, Apr. 10, 1996, known are a process and plant for the production of methanol. The process comprises a separate feeding of compressed, (and then heated) hydrocarbon-containing (natural) gas, and compressed air or oxygen, to the reactor mixing zone (mixing unit); a sequential stepwise oxidation of hydrocarbon-containing gas (at the original temperature of 325-50000, a 3-10 MPa pressure and the oxygen content of 1.5-8% by volume) in the reactor's two reaction zones, with an additional feeding of oxygen or air to the next mixing zone; cooling the reaction mixture; separating methanol from the cooled gas-liquid mixture; and sending exit gases to the original hydrocarbon-containing gas (pipeline) or for burning.

The methanol production plant comprises a compressor; a recuperative heat exchanger and a combustion heater (for compressing and heating hydrocarbon-containing gas which are connected to the source of hydrocarbon-containing gas); a separate source of oxygen or air connected to the compressor; a reactor with two mixing and reaction zones (with pipelines for the separate feeding of hydrocarbon-containing gas, and air or oxygen; to the mixing zones); a recuperative tubular heat exchanger (sequentially installed and connected to the reaction zone of the reactor) for cooling the reaction mixture and heating cold hydrocarbon-containing gas; a cooler-condenser; a separator for separating methanol from the cooled gas-liquid reaction mixture; and a pipeline for feeding exit gases to the hydrocarbon-containing gas pipeline or to the reactor. The known process and plant do not provide a sufficiently high rate of methane conversion during one pass of natural gas through the reactor. For an effective recycling of gases, it is practically mandatory to use oxygen as the oxidizer, and its use increases the product's cost by 20-30%. In addition, the relatively high original reaction temperature and subsequent self-heating of the reaction mixture reduces the yield of the desired product and contributes to intensive sooth formation, which complicates the operation of production equipment and reduces the quality of the resulting methanol.

The process and plant that are the closest analogues of the claimed invention are described in RU No. 2162460 (published Jan. 27, 2001). The known plant for homogenous oxidation of natural gas comprises a source of natural gas, heat exchangers, reactors, a separator, receivers, a source of oxygen supply for conducting gas-phase oxidation of gas, and means for cooling the reaction mixture, which, are interconnected by means of the main pipeline. The known process for homogenous oxidation of natural gas comprises a separate feeding of compressed (and then heated) natural gas and oxygen-containing gas, and oxidizing it with oxygen in reactors (with subsequent cooling/quenching), wherein the process is characterized by the fact that the reaction mixture is cooled by 70-1500 C before each subsequent stage of oxidation and quenched in the last reaction zone, thereby reducing the reaction mixture temperature at least by 2000 C over the time shorter than 0.1 of its dwell time in the reaction zone. The process is conducted cyclically, performing cycled gas purging cycles and cycle completion. The disadvantage of the known plant is its low efficiency, and the disadvantage of the process is a lower yield of the resulting product and contamination of cycled gas with products of sulfur oxidation (SO2). In the proposed process and plant, several reactors are sequentially installed. The thermal effect of reactions in them is used for generating steam in waste-heat boilers, and reaction gas is cooled to the optimum temperature of entry into the next reactor, which makes it possible to achieve a higher rate of converting it to methanol, formaldehyde and ethanol. The presence of an increasing percentage of sulfur oxides in the cycled gas (SO2) in the known plant substantially reduces the rate of reaction gas conversion because sulfur oxides are not an inert gas, and in this case, they act as an anti-catalyst.

So the technical result of the invention is increased utilization efficiency, higher yield of the resulting product, and cleaning the cycled gas to prevent it from accumulating SO2. In addition, the invention expands the arsenal of processes and plants for homogenous oxidation of methane-containing gas. The rate of reaction gas conversion to products of homogenous oxidation in one reactor is low (minimum 2%, maximum 4%, depending on the gas composition and selected optimum temperature and pressure). Therefore, from an economical standpoint, the minimum number of reactors can be three, and their maximum number would be determined by the original composition of the gas. Also, it is most efficient from a process standpoint to connect the outlet of the last absorber (after purifying cycled gas from SO2, CO2 and CO) to the main pipeline.

### SUMMARY

Said technical result is achieved in the proposed plant due to the fact that the plant uses the following, which are sequentially connected by means of a main pipeline: (i) a source of methane-containing gas, heat exchangers, and at least three reactors made of carbon steel, wherein each reactor, except the last one, is connected independently to waste-heat boilers and the reactors are also connected to oxygen supply sources; (ii) a separator, wherein, in the path from the last reactor,—which is not connected to the waste-heat boilers—to the separator, there are heat exchangers which heat the cycled gas and provide generation of water vapor which is combined with vapor from waste-heat boilers by means of an additional pipeline that feeds steam to a rectifying tower for separating the end products; (iii) pipelines, for feeding the liquid phase from the separator to the rectification stage where rectified methanol, ethyl alcohol and formaldehyde are produced; and (iv) sequentially installed absorbers, to which the gas phase is fed for cleaning it of SO2, CO2 and CO where absorber liquid phase outlets are connected to receivers which makes regeneration of solutions with separation and removal of CO and CO2 fractions possible. The last absorber outlet is connected to the main pipeline before the point of location of the cycled gas blower which makes partial purging of the circulation cycle for removal of existing inert gases possible.

In the proposed process, the technical result is achieved due to the fact that methane-containing gas preheated to 430-4500 C is fed to at least three sequentially installed oxidation reactors made of carbon steel, wherein each reactor, except the last one, is independently connected to waste-heat boilers. The reactors are also fed with oxygen in an amount such as to form a mixture below the explosive limit of concentration. This induces a homogeneous oxidation of methane-containing gas and a simultaneous increase of the gas mixture temperature to 540-5600 C, with subsequent rapid quenching-cooling of the gas mixture to 440-4500 C in waste-heat boilers. There, steam is formed which is fed to the rectifying tower for the separation of end products. Then the reaction mixture is fed from the last reactor (which is not connected to a waste-heat boiler) to the separator wherein, on the path to the separator, the reaction mixture heats the cycled gas, and part of the mixture heat is used for generating water vapor. The water vapor is combined with steam from waste-heat boilers. From the separator, the liquid phase is fed to the rectification stage where rectified methanol, ethyl alcohol and formaldehyde are produced. The gas phase involves sending gas for cleaning from SO2, CO2 and CO. At the same time the gas phase is cleaned, partial purging cycles of the circulation cycle are performed to remove inert gases, for instance, nitrogen and argon. The number of purging cycles is determined based on the permissible amount of inert gases in the cycle. After cleaning, a purging the cycle is completed by replenishing the gas phase/cycled gas with the original methane-containing gas and re-feeding the newly formed gas to the reactor.

### FIGURES

FIG. 1 schematically shows the plant diagram.

### THE DIAGRAM LEGEND

1—the cycled gas line;
2—makeup gas;
3, 4, 5—heat exchangers;
6—the heat exchanger-condenser;
7, 8, 9—homogenous oxidation reactors;
10—the oxidate separator;
11—the oxidate receiver;
12—the oxidation products rectification unit;
13—oxygen supply;
14, 15—waste-heat boilers;
16, 17—absorbers with vortex packing;
18—the pipeline for regenerated heat in the form of steam;
19—feed water;
20—cooling water;
21—the cycled gas blower;
22—absorber and desorber oxidate pumps;
23—methanol fraction supply;
24—start gas;
25—CO and CO2 fractions;
26—the desorber;
27—the heat exchanger-absorbent cooler;
28—the regenerative heat exchanger;
29—the heat exchanger-boiler;
30, 31—absorbing solution receivers

### DESCRIPTION

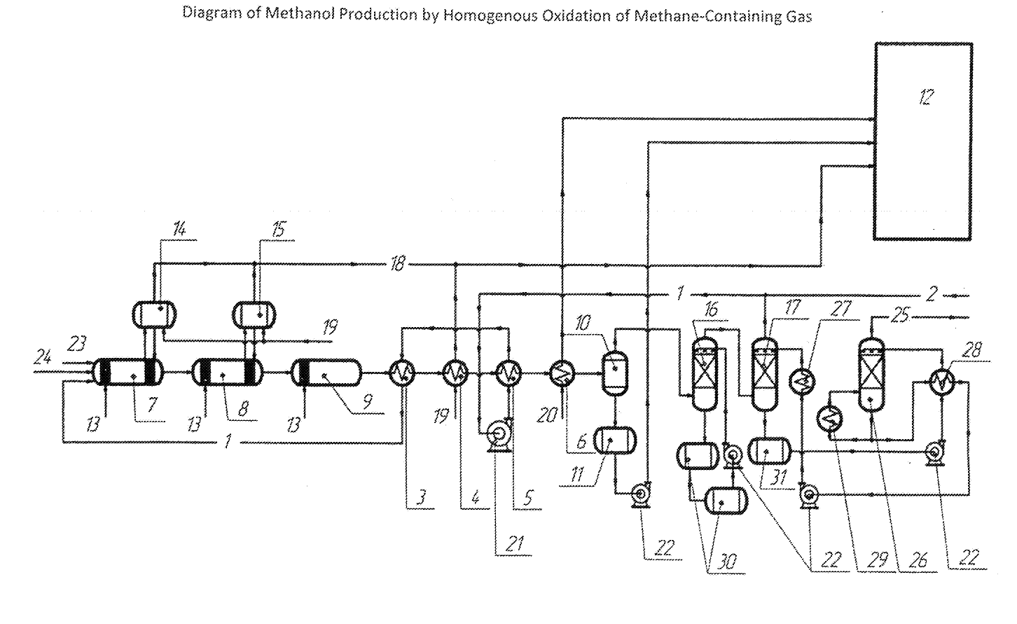

The plant for homogenous oxidation of methane-containing gas shown in FIG. 1 comprises the following, which are connected by means of the main pipeline 1: the methane-containing gas source 2, heat exchangers 3, 4, 5 and 6, sequentially installed reactors 7, 8 and 9, the separator 10, receivers 11; 30 and 31, the oxygen supply source 13 for conducting non-catalytic gas-phase oxidation of gas, and means for cooling the reaction mixture. The plant also comprises waste-heat boilers 14 and 15, absorbers 16 and 17, the additional pipeline 18 connecting outlets of the waste-heat boilers 14 and 15 and an outlet of the heat exchanger 4 for using the steam that has formed in the process at the stage of products rectification of homogenous oxidation reaction, wherein in order to eliminate partial decomposition of products of homogeneous oxidation, reactors 7, 8 and 9 which are connected to the oxygen supply source 13 are made from carbon steel.

The heat exchanger 3 is made with the ability to heat the cycled gas, its inlet is connected to the outlet of the heat exchanger 5, and the outlet of the latter is connected to the cycled gas blower 21, wherein the outlet of the heat exchanger 3 is connected to the inlet of the reactor 7, which makes the formation of primary products of homogenous oxidation of methane-containing gas possible. Reactors 7, 8 and 9 are connected to each other sequentially by means of the main pipeline 1, which makes the formation of secondary products of homogenous oxidation of gas possible. When heat (received by cooling water from the cooling water pipeline 20) passes through the heat exchanger 6, it is used for heating oxidation products when they are fed to the first rectifying tower (not shown) of the unit 12 for separating oxidate into its components.

The outlet of the last reactor 9 is connected to the second inlet of the heat exchanger 3 for additional heating of the gas fed to it from the source 2 of methane-containing gas to 430-4500 C.

Heat exchangers 3, 4, 5 and 6 are connected to each other sequentially. The last heat exchanger 6 is made with the ability for final cooling and condensation of oxidation products. Its inlet is connected to the cooling water pipeline 20, and its outlet is connected to the separator 10 inlet. One of the outlets of the separator 10 (the liquid condensate outlet) is connected to one of the receivers 11 for subsequently pumping condensed products from the receiver by means of the blower 22 to the rectification stage for producing rectified methanol, ethyl alcohol and formaldehyde. The second outlet of the separator 10 (the non-condensed gases outlet) is connected to an inlet of the first absorber 16, which is able to clean the cycled gas of SO2. Then, gases from the absorber 16 are fed to the inlet of the second absorber 17, which makes it possible to clean the cycled gas of CO and CO2 fractions.

Liquid phase outlets of absorbers 16 and 17 are connected to receivers 30 and 31, respectively, which makes regeneration of absorbing solutions possible with separation and removal of CO and CO2 fractions from the desorber 26. Outlets of the receivers 30 and 31 are connected to inlets of the absorbers 16 and 17 to feed the solutions that have formed for cleaning the cycled gas of SO2, CO and CO2. The outlet of the second absorber 17 is connected to the pipeline 1 before the point of location of the cycled gas blower, which makes partial purging of the circulation cycle for removal of existing inert gases possible.

The means for cooling the reaction mixture are heat exchangers 3, 4, 5 and 6 and waste-heat boilers 14 and 15. The feed water source 19 is connected to waste-water boilers 14 and 15. For steam generation, it is also connected to the heat exchanger 4. Item 23 indicates the methanol fraction supply. Item 24 indicates the start gas supply. Item 25 indicates the outlet of the CO and CO2 fraction for utilization. Items 30 and 31 indicate vessels for absorbing solutions. Item 22 indicates blowers. Item 27 indicates the heat exchanger-cooler. Item 28 indicates the regenerative heat exchanger, Item 29 indicates the heat exchanger-heater.

The plant for homogenous oxidation of methane-containing gas and the process for homogenous oxidation work as follows. Based on the condition of reducing power consumption for the compression of methane-containing gas with recycling, the process of homogenous oxidation of methane-containing gas resulting in methanol production is conducted as follows: cycled gas heated to 4500 C in the heat exchanger 3 is fed to the reactor 7, where it is mixed with oxygen in the ratio that is lower than the explosive limit. The oxygen partially oxidizes methane and other hydrocarbons contained in the gas. In the process, the gas mixture temperature increases to 540-5600 C, and methanol, ethanol and formaldehyde are formed. The reaction of homogeneous oxidation products formation is reversible. Consequently, it is necessary to perform rapid quenching-cooling of the reaction mixture from 540-5600 C to 440-4500 C. This is done in the waste-heat boilers 14 and 15 and the heat exchanger 3.

In general, the presence of nickel and its compounds (stainless steels) in the reaction zone results in partial decomposition of homogenous oxidation products. To prevent this phenomenon, reactors 7, 8 and 9 are made from carbon steel.

After the reactor 7, the cycled gas is fed to the reactor 8 where the process of methane homogenous oxidation is repeated, similar to the process in the reactor 7. Then the similar process is performed in the reactor 9. While the cycled gas heats in the heat exchanger 3, the gas is fed to the reactor 7. The remaining heat potential of the cycled gas is removed in the heat exchanger 4 to generate steam that is used, together with the steam from the waste-heat boilers 14 and 15, at the stage of rectification of homogeneous oxidation products. After the heat exchanger 4, the cycled gas in the heat exchanger 5 heats the gas fed through the heat exchanger 3 to the reactor 7. Final cooling and condensation of oxidation products is conducted in the heat exchanger 6 using cooling water. Condensed products of homogenous oxidation are separated in the separator 10 and moved to the receiver 11. From there, they are fed to the rectification stage where they are distilled, and rectified methanol, ethyl alcohol and formaldehyde are produced. The cycled gas from the separator 10 is fed to the absorbers 16 and 17 where it is washed—cleaned of SO2 in the first absorber and of CO and CO2 in the second absorber. The absorption solution of the second absorber is regenerated in the desorber 26. CO and CO2 in it are separated, and the solution (cooled in the heat exchanger 27) is fed again for cleaning the cycled gas.

The separation of CO and CO2 from the gas recycle is necessary, and is based on a 15% consumption reduction of oxygen used for reoxidation of CO to CO2 and a reduction in the amount of inert gases SO2 and CO2 in the cycle.

Then, partial purging of the circulation cycle to remove inert gases that entered the cycle together with methane-containing gas and oxygen (nitrogen, argon, krypton, etc.) is provided. The number of purging cycles is determined from the balance based on the permissible amount of inert gases in the cycle. The amount of inert gases in the cycled gas can be as high as 70%. A further increase in the amount of inert gases results in a decreased rate of reaction gas conversion. After a cycle is purged, it is replenished with methane-containing gas. Then, the blower 21 feeds the cycled gas to the heat exchangers 5, 4 and 3 and to the inlet of the reactor 7. Thus, the entire operating cycle of the invention has been described.

### Comparison Table 1

### The Reactant Gas Rate of Conversion, %

### Known Process and Plant 6.0

### Proposed Process and Plant 9.0

Table 1 shows the data on the rate of conversion during one cycle in a process design with three sequentially installed reactors based on experimental data produced in one reactor.
Thus, the invention increases operating efficiency and the yield of the manufactured product.

The invention can be used in the petrochemical industry to use flare gases in production of methanol, formaldehyde and other products.

1. A process for homogenous oxidation of methane-containing gas comprising the feeding of methane-containing gas preheated to 430-4500 C to at least three sequentially installed oxidation reactors made of carbon steel, wherein each reactor, except the last one, is connected independently to waste-heat boilers, and the reactors are also fed with oxygen in an amount such as to form a mixture below the explosive limit of concentration, which induces homogenous oxidation of methane-containing gas with a simultaneous increase of the gas mixture temperature to 540-5600 C, with subsequent rapid quenching-cooling of the gas mixture to 440-4500 C in waste-heat boilers where steam is formed, the steam is fed to a rectification tower for separation of end products, then from the last reactor which is not connected to a waste-heat boiler the reaction mixture is fed to a separator wherein, on the way to the separator, the reaction mixture heats the cycled gas, and some of its heat is also used for the generation of water steam which is combined with the steam from waste-heat boilers, from the separator the liquid phase is fed to the rectification stage where rectified methanol, ethyl alcohol and formaldehyde are produced, and the gas phase is fed for cleaning it from SO2, CO and CO2, wherein, at the same time the gas phase is cleaned, the circulation cycle is partially purged to remove inert gases, for instance, nitrogen and argon, the number of purging cycles is determined based on the permissible amount of inert gases in the cycle, and after cleaning and purging the cycle is completed by replenishing the gas-phase/cycled gas with the original methane-containing gas and feeding the newly formed gas to the reactor.

2. The plant for the realization of the process according to claim 1 comprising the source of methane-containing gas, heat exchangers and at least three reactors made of carbon steel which are sequentially connected to each other by means of a main pipeline, wherein each reactor, except the last one, is independently connected to waste-heat boilers, the reactors are also connected to oxygen supply sources and the plant also comprises a separator, wherein, on the pass to the separator from the last reactor which is not connected to waste-heat boilers, there are heat exchangers that heat the cycled gas and also provide generation of water steam, which is combined with steam from waste-heat boilers by means of an additional pipeline that feeds steam to a rectification tower for separation of end products, pipelines for feeding the liquid phase from the separator to the rectification stage where rectified methanol, ethyl alcohol and formaldehyde are produced, and the gas phase is fed for cleaning it from SO2, CO and CO2 to sequentially installed absorbers where the liquid phase outlets of the absorbers are connected to receivers which makes possible regeneration of solutions with separation and removal of CO and CO2 fractions, and the outlet of the last absorber is connected to the main pipeline before the point of location of the cycled gas blower which makes possible partial purging of the circulation cycle for removal of existing inert gases.
